(19) United States
(12) Patent Application Publication
(10) Pub. No.: US 2012/0190879 A1
(43) Pub. Date: Jul. 26, 2012

(54) HYDROCHLORINATION OF ELECTRON-DEFICIENT ALKENES

(76) Inventor: Michael Todd Coleman, Batesville, AR (US)

(21) Appl. No.: 13/012,436

(22) Filed: Jan. 24, 2011

(51) Int. Cl. C07C 255/03; C07C 233/13; C07C 53/15; C07C 17/02

(52) U.S. Cl. 558/460; 570/261; 564/209; 562/602

(57) ABSTRACT

The present invention pertains to a method for the hydrochlorination of electron deficient alkenes, particularly alkenes having the functional groups COOH, CONH₂, and CN. Specific alkenes discussed include acrylic acid, crotonic acid, methacrylic acid, acrylonitrile, acrylamide, and methacrylonitrile. The alkene is combined with a primary or secondary alcohol (e.g., isopropanol) and an acid chloride (e.g., acetyl chloride) under conditions suitable to chlorinate the alkene. Products formed by the invention include 3-chorosubstituted carbonyl compounds such as 3-chlorpropionic acid (3-CPA), 3-chloropropionamide (3-CPAD), and 3-chloropropionitrile among other products.

### CROSS REFERENCE TO RELATED APPLICATION

Not applicable.

### FIELD OF THE INVENTION

The present invention pertains to the hydrochlorination of electron deficient alkenes. In particular, the present invention pertains to the hydrochlorination of electron deficient alkenes using hydrochloric acid (HCl) that is generated in-situ from a reaction mixture containing acid chloride and an alcohol. More specifically, the invention pertains to a novel process for the preparation of various chemical compounds from the hydrochlorination of acrylic acid. Representative chemical compounds prepared by the process according to the invention include 3-chloropropionic acid and 3-chloropropionamide.

### BACKGROUND OF THE INVENTION

The invention disclosed and claimed herein pertains to the hydrohalogenation, specifically the hydrochlorination, of alkenes. The hydrohalogenation of alkenes is one of the first reactions taught in introductory organic chemistry classes. Basic principles such as nucleophilicity, carbocation stability, and solvent effects are learned from these simple transformations. The examples taught typically include the addition of either anhydrous HCl or anhydrous HBr to an alkene such as isobutylene or substituted cyclohexenes in a solvent based system.

While these reactions are very simplistic in an educational sense, reducing the underlying chemistry to practice on an industrial scale can be challenging. Challenges include accurate methods to meter or measure amounts of dangerous anhydrous gas, proper selection and maintenance of piping to avoid leaks and minimize safety incidents, and economic challenges due to the capital intensity of handling bulk anhydrous HCl. In addition to being difficult and dangerous to handle, anhydrous HCl is a rather expensive raw material.

Unfortunately, hydrochlorination reactions are critical to several chemical processes and industries. For example, 3-chlorosubstituted carbonyl compounds such as 3-chloropropionic acid (3-CPA) and 3-chloropropionamide (3-CPAD) are used as raw materials in the manufacture of biocides, colorants, pharmaceutical products, cosmetic products and additives for plastics.

With respect to pharmaceutical products, the use of 3-chlorosubstituted carbonyl compounds as synthons can be found in several classes of compounds with medicinal applications. One such class of compounds are chromanones such as sorbinil which is an aldose reductase inhibitor. Another such compound, Gö6976 is an indolecarbazole that has been identified as an inhibitor of protein kinase C and acts in arresting the replication of DNA-damaged cells. Beclamide is a proprionate that has long been known to function as an anticonvulsant and is more recently being explored in the area of behavior modification.

Given the importance of these drugs and the use of 3-chlorosubstituted carbonyl compounds in other specialty chemicals, there is a need for an efficient and safe method for producing 3-chlorosubstituted carbonyl compounds. Unfortunately, current production methods are neither efficient nor as safe as they could be.

When considering 3-CPA, the initial preparation reported in the literature makes use of less than desirable starting materials, including acrolein, fuming nitric acid, ethylene cyanohydrin, and cyanide. More recent methods of preparing 3-CPA have focused on either the treatment of aqueous acrylic acid with anhydrous HCl or acrylonitrile with anhydrous HCl followed by hydrolysis. The hydrohalogenation of alkenes and alkynes with silica gel or alumina by HX precursors such as thionyl chloride or acetyl bromide have been reported. The preparation of 3-chloropropionates from alkyl acrylates, anhydrous alcohols, and acetyl chloride in a solvent system has also been discussed.

The above methods for preparing 3-CPA and other hydrohalogenated alkenes suffer from various flaws. Solvent based systems require additional (and costly) steps to remove the solvent. Methanol/acetyl chloride systems can generate methyl chloride which is heavily regulated (and costly) air pollutant under the United States Clean Air Act. Accordingly, there is a need for a method of hydrochlorinating alkenes that is more efficient, less costly, and safer than known production methods.

### OBJECT OF THE INVENTION

It is therefore one object of the present invention to provide a method for the hydrochlorination of alkenes that avoids the use of expensive and dangerous anhydrous hydro-halogens.

It is a further object of the invention to provide a safe and efficient method for the hydrochlorination of alkenes that substantially or completely eliminates the use of aqueous reaction mixtures and solvents.

It is a still further object of the invention to provide method for the hydrochlorination of alkenes that utilizes the in situ production of HCl rather than the use of anhydrous HCl.

### BRIEF SUMMARY OF THE INVENTION

The claimed invention meets these and other objects by providing a method of hydrochlorination of alkenes utilizing in situ generation of HCl. The method generally comprises the steps of forming a reaction mixture containing an alkene, an alcohol, and an acid chloride and maintaining the reaction mixture at temperature sufficient and for a time sufficient to chlorinate the alkene.

More specifically, the method according to the invention comprises selecting an alkene from the group consisting of acrylic acid, crotonic acid, methacrylic acid, acrylonitrile, acrylamide, and methacrylonitrile, and adding to it a primary or secondary alcohol having I to 4 carbon atoms. In preferred embodiments the alcohol is isopropanol. An acid chloride is then added to the alkene and alcohol to complete the reaction mixture. The reaction mixture is then kept below about 30° C. and allowed to react for a time sufficient to chlorinate the alkene.

Products formed by the invention include 3-chorosubstituted carbonyl compounds such as 3-CPA, 3-CPAD, among other products.

### BRIEF DESCRIPTION OF THE DRAWINGS

FIG. 1 is a chart representing the relative amounts of reactants and products that are present in a reaction mixture over the course of a reaction.

FIG. 2 is a proposed reaction scheme for the data shown in FIG. 1.

### DETAILED DESCRIPTION

In the following description, for purposes of explanation, numerous details are set forth to provide an understanding of one or more embodiments of the present invention. Furthermore, the following detailed description is of the best presently contemplated mode of carrying out the invention based upon the existing experimental data. The description is not intended in a limiting sense, and is made solely for the purpose of illustrating the general principles of the invention.

As used herein, the term electron withdrawing group or “EWG” means a chemical substituent that draws electrons to it. Specifically, the term electron withdrawing group as used herein includes COOH (carboxylic acids), CONH₂ (amides), and CN (nitriles).

The term non-aqueous reaction mixture means a reaction mixture that is initially formed without the presence of significant amounts of water (i.e., anhydrous materials) but includes reaction mixtures in which water is formed as part of the overall reaction process.

The term solvent means an organic liquid that is employed to dissolve an alkene utilized in the practice of the invention. The term solvent as used herein does not include alcohols.

The method according to the invention utilizes and expands the use of in situ generated HCl in the hydrochlorination of alkenes. This approach to hydrochlorination avoids the physical handling and economic challenges previously discussed with systems that utilize anhydrous HCl.

Initially, several options to develop such an “in situ HCl” method appeared available.

Exposure of chlorinated inorganic reagents such as thionyl chloride, phosphorous trichloride, or phosphorous oxychloride to a hydroxy compound such as water or an alcohol would lead to the in situ production of HCl. However, these materials produce by-products that are undesirable or would be difficult to remove from a reaction mixture.

Another option was to consider utilizing acid chloride and an alcohol in a reaction mixture. An acid chloride and an alcohol can react to produce HCl in situ. Furthermore, the primary by-product of the reaction between an acid chloride and an alcohol is a volatile ester which should be easily removed by distillation.

The method according to the invention centers on the hydrochlorination of unsaturated compounds using HCl produced in situ via the reaction of an acid chloride and an alcohol. This approach has several advantages over previously described methods: 1) readily available, easy to handle raw materials; 2) the reaction is performed in a non-aqueous reaction mixture thus eliminating the need to remove water and avoiding the potential formation of hydrates; 3) isolation and purification of solid products by crystallization; 4) use of as little as a 10% excess of HCl generated in situ from the alcohol and acid chloride is realized with some substrates, greatly minimizing raw material cost, 5) produces a by-product alcohol acetate that can be recovered, purified, and marketed to help off-set the cost of production, and 6) may be practiced in the absence of a solvent or other additives such as polymerization blockers.

In very broad terms, the method according to the invention is a method for the hydrochlorination of alkenes utilizing the in situ generation of HCl in the reaction mixture rather than the introduction of anhydrous HCl to the system. The method comprises the steps of forming a reaction mixture which comprises an alkene, an alcohol, and an acid chloride. Preferably, the reaction mixture is non-aqueous and need not contain a solvent (e.g., methyl acetate, ethyl acetate, propyl acetate, ethyl formate, dichloromethane, tricholoromethane, carbon tetrachloride, 1,2-dichloroethane, or benzene). Preferred embodiments utilize a solvent free or substantially solvent free reaction mixture. As used herein the phrases “solvent free” and “substantially solvent free” mean that the reaction mixture does not contain solvents for the purpose of dissolving the alkene that is to be chlorinated. The reaction mixture is then maintained at a temperature sufficient to achieve hydrochlorination of the alkene.

The alkene that may be utilized in the practice of the invention may be any alkene having an electron withdrawing group (EWG) having the general formula

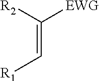

where R₁ is selected from the group consisting of H and CH₃ and R₂ is selected from the group consisting of H and CH₃, provided that R₁ and R₂ are not both CH₃. The EWG may be any group that draws away electrons from the carbon-carbon double bond but is preferably selected from the group consisting of COOH, CONH₂, and CN where R is an alkyl group having 1 to 4 carbon atoms. In preferred embodiments the alkene is selected from the group consisting of acrylic acid, acrylonitrile, methyacrylonitrile, and acrylamide.

Other alkenes suitable for use in the practice of the invention include substituted acrylic acids such as crotonic acid and methacrylic acid.

The alcohols suitable for use in the method according to the invention include primary and secondary alcohols having 2 to 4 carbon atoms. In preferred embodiments the alcohols are selected from the group consisting of ethanol, propanol, isopropanol, butanol, and isobutanol. Isopropanol is the preferred alcohol and is used in the majority of the examples discussed herein.

Essentially any acid chloride may be used in the practice of the invention. The primary qualifier on the choice of acid chloride is that the corresponding alcohol ester should be a liquid. The chemical reactions describing this relationship are discussed below. Generally speaking, the larger the acid chloride the more difficult it is to remove the resulting ester. Accordingly, in preferred embodiments the acid chloride has the general formula

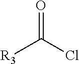

where R₃ is an alkyl group having 1 to 4 carbon atoms. Acetyl chloride (R₃═CH₃) is a preferred chloride and is used in the majority of the examples discussed herein. Furthermore, the acetyl chloride is typically added in a stepwise fashion to a reaction mixture that already contains the alkene and alcohol.

Once the reaction mixture containing the acid chloride is formed, the reaction mixture is then maintained at a temperature that is sufficient to chlorinate the selected alkene. Typically, hydrochlorination reactions are maintained at 30° C. or less and the method according to the invention utilizes this temperature range. Preferably, the reactions are conducted with cooling such that the reaction temperature is maintained at 20° C. or less, most preferably at 15° C. or less. Specific temperature conditions are discussed in the examples that follow.

The reaction is also maintained for a time sufficient to chlorinate the alkene. For acrylic based alkenes the resulting hydrochlorinated alkene will have the general formula

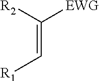

where R₁, R₂, and EWG are as described previously.

As will be discussed in more detail below, preferred products of the method according to the invention include 3-chloropropionic acid, 3-chloropropionamide, 3-chloropropionitrile, 3-chloro-2-methyl propionic acid, 3-chloro-2-methyl propionitrile, and 3-chlorobutanoic acid.

Turning now to more specific embodiments of the method according to the invention, initial attempts to develop an “in situ” HCL process utilized methanol as the alcohol combined with the acid chloride to generate HCl. Acrylic acid served as the alkene and acetyl chloride served as the acid chloride in these initial tests. Although the reaction resulted in the desired product (3-CPA), an equivalent of methyl ester was formed. While the methyl ester was not desired, the target acid could be generated by hydrolysis of the ester. This would, however, result in an additional processing step as well as having to isolate the product from water.

Another problem in the use of methanol as the alcohol is that there exists a strong possibility of generating methyl chloride. Methyl chloride is a heavily regulated air pollutant under the U.S. Clean Air Act. The generation of methyl chloride may require emission control, emission testing, and periodic reporting, thus significantly increasing the cost of manufacturing.

The high level of methyl ester formation and potential environmental issues encouraged the search for another alcohol. t-Butanol and isopropanol were also examined as potential alcohols for the process. It was found that when t-butanol was used in the process, the HCl that was generated reacted faster with t-butanol to form t-butyl chloride than with the alkene (in this case, acrylic acid) to form 3-CPA.

Isopropanol, however, was found to provide the appropriate reactivity for the system under investigation. No evidence of isopropyl chloride formation was detected. While some ester was formed, it was at a level that was easily removed during product purification via crystallization. Based on these results and the successful use of methanol, it is believed that primary and secondary alcohols having 1 to 4 carbon atoms can be utilized in the method according to the invention.

Although Applicant does not wish to be bound by any particular theory regarding the reaction process underlying the claimed invention, the following proposed explanation is offered as an aid to the reader. Using acrylic acid as the alkene, acetyl chloride as the acid chloride, and a primary or secondary alcohol having 1 to 4 carbon atoms, it is believed that the hydrochlorination of acrylic acid is generally represented by the following reaction. Hydrochlorination of acrylic acid to form 3-CPA

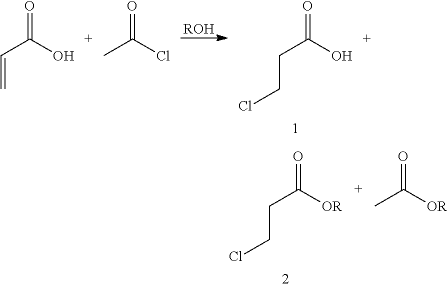

The “1” and “2” represent the acid and ester products, respectively, that were seen at the completion of the reaction. The following table reflects the relative percentages of acid and ester generated during three test runs using methanol, isopropanol and t-butanol as the alcohol respectively. As noted previously, the tertiary alcohol t-butanol did not result in formation of the desired product.

TABLE 1

| R | 1 | 2 |
|---|---|---|
| methanol | 63 | 37 |
| isopropanol | 94 | 6 |
| t-butanol | 0 | 0 |

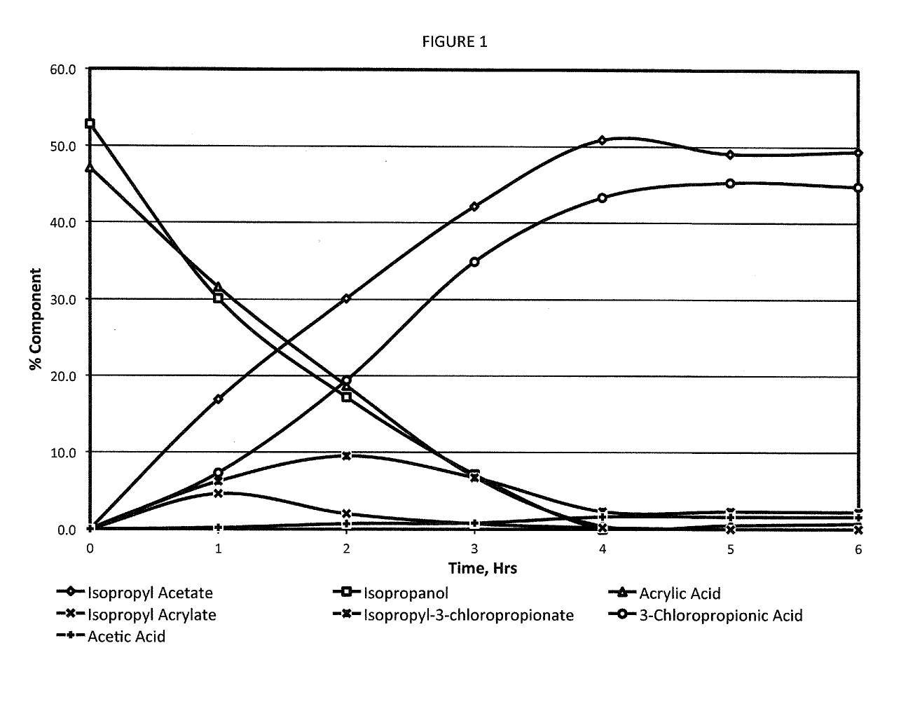

To gain a better understanding of the overall reaction process, another representative reaction was conducted using acrylic acid as the alkene, isopropanol as the alcohol, and acetyl chloride as the acid chloride. Samples of the reaction mixture were taken at various intervals during a stepwise addition of acetyl chloride. One-fourth of the total acetyl chloride charge was made over thirty minutes and then allowed to stir for thirty minutes before sampling. This was repeated three additional times for a total of four samples. The reaction mixture was stirred for one hour and maintained at less than 15° C. The reaction mixture was then warmed to room temperature over an hour and sampled again. The results are shown in FIG. 1.

As shown in FIG. 1, several different compounds were identified in the reaction mixture as the reaction progressed. The reactants (isopropanol, acrylic acid, acetyl chloride) were identified along with the target product 3-CPA. However, isopropyl acetate, isopropyl-3-chlorpropionate, acetic acid, and isopropyl acrylate were also identified.

As expected, the concentration of isopropanol and acrylic acid decreased in a linear manner as acetyl chloride was added. The concentration of isopropyl acetate and 3-CPA increased with the increasing amount of acetyl chloride.

It is interesting to note that concentrations of the esters isopropyl acrylate and isopropyl-3-chloropropionate initially build to a maximum concentration within the first 25-50% of the acetyl chloride addition. As the addition of acetyl chloride continues to completion the levels drop and stabilize, with isopropyl acrylate being reduced to non-detectable levels.

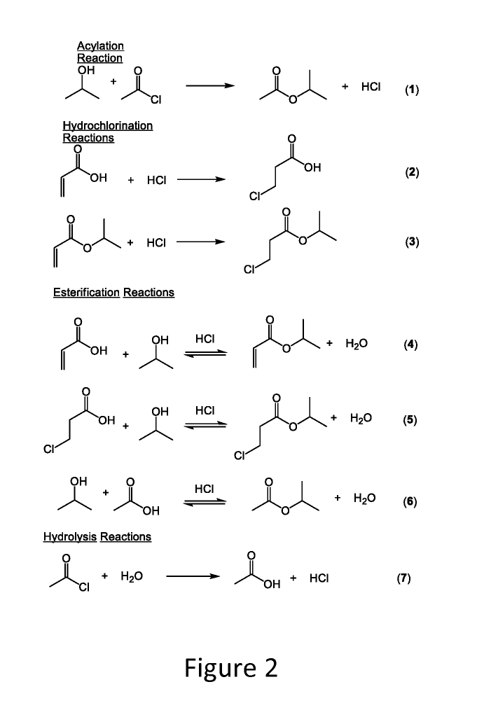

This data set indicates that a series of complex reactions are likely taking place to perform this simple hydrochlorination. FIG. 2 is a proposed reaction scheme to aid in explaining the data shown in FIG. 1. This reaction scheme is presented for explanatory purposes only and is based on current data. It is not Applicant's intent to be bound by the proposed reaction scheme as additional research may shed further light on this process.

The data from FIG. 1 indicates that in addition to the acylation of isopropanol (Eq. 1) and the hydrochlorination reactions (Eq. 2 & 3), a hydrolysis reaction (Eq. 7) and a trio of esterification equilibriums (Eq. 4, 5, & 6) must be considered.

The data suggests that after the initial acylation of isopropanol (Eq. 1), the HCl that is generated participates in three different reactions. One reaction is the desired hydrochlorination of acrylic acid to form 3-CPA (Eq. 2). The other two reactions are the acid-catalyzed esterification of acrylic acid (Eq. 4) and 3-CPA (Eq. 5), producing water as a by-product. The ester of acrylic acid (product of Eq. 4) can participate in the hydrochlorination reaction (Eq. 3). Water is available to consume acetyl chloride, producing acetic acid and HCl (Eq. 7). The conversion of isopropyl-3-chloropropionate to 3-CPA occurs via hydrolysis (Eq. 5) or transesterification with acetic acid. Since both acetic acid and isopropyl-3-chloropropionate are present at the end of the reaction, this suggests the hydrolysis reaction is the operative mechanism.

### Hydrochlorination of Other Acrylic Functional Groups

The use of other EWG was explored. In particular, there was interest in observing the reactivity of acrylic functional groups such as amides, nitriles, and esters. To accomplish this task, reactions were run in which acrylonitrile, and acrylamide served as the alkene. Isopropanol served as the alcohol and acetyl chloride served as the acid chloride. The alkene and the isopropanol were mixed in a reaction vessel and the acetyl chloride added in a stepwise fashion as before under standard conditions (e.g., 1.1 eq HCl at less than 15° C. for two hours then 16 hours at ambient temperature—see examples for specific quantities of reactants). Treatment of these compounds under these conditions resulted in acrylonitrile expressing similar rates of reaction as acrylic acid. Acrylamide, however, was less reactive than the other two compounds (Table 2).

TABLE 2
Relative reaction rate and yield of acrylic functional groups

| | Relative | |
|---|---|---|
| Compound | Reaction Rate | Yield |
| Acrylic Acid | 1.0 | 85.5 |
| Acrylonitrile | 0.96 | 84.3 |
| Acrylamide | 0.59 | 47.4 |

While amides are not considered strong bases, they do possess some basicity. It was theorized that perhaps the basicity of acrylamide neutralized some of the HCl generated in the system. Another reaction was run increasing the HCl to two equivalents (e.g., the quantity of isopropanol and acetyl chloride was doubled). A 98% conversion of acrylamide to 3-chloropropionamide was realized in 82.6% yield.

### Hydrochlorination of Substituted Acrylic Acids

While the initial interest was in the hydrochlorination of acrylic acid, it was of interest to determine how substituted alkene acids performed in this system. Readily available acids of this type include methacrylic acid, crotonic acid, and cinnamic acid. Each acid was subjected to standard hydrochlorination reaction conditions (1.1 eq “HCl” @ <15° C. with isopropanol; two hours at <15° C. followed by 16 hours at ambient temperature). The study demonstrated the following order of reactivity as discerned by percent conversion of the starting acid and the product yield: acrylic acid >crotonic acid>>methacrylic acid>>cinnamic acid. Table 3 summarizes the data.

TABLE 3
Relative reaction rate and yield of substituted acrylic acids

| Acid | Relative Reaction Rate | Yield |
|---|---|---|
| Acrylic | 1.0 | 85.5 |
| Crotonic | 0.93 | 65.4 |
| Methacrylic | 0.53 | 37.2 |
| Cinnamic | 0.0 | 0.0 |

Standard conditions of 1.1 eq. HCl @ <15° C. for two hours then 16 hours at ambient temperature; isopropanol as the alcohol.

Current data indicates that there are at least two potential mechanisms to consider for the hydrochlorination of electron-deficient alkenes. Again, Applicant does not intend to be bound by the following theories but presents them in an effort to fully describe the invention. One mechanism would be the nucleophilic attack of the alkene on the hydronium ion to form a carbocation, which would then be captured by the chloride ion. Another potential mechanism would be a Michael-type addition of chloride to the activated alkene, followed by protonation of the resulting enolate.

If the nucleophilic mechanism were in operation, one would expect to find 2-chloro-2-methyl propionic acid as a resulting product. This material should be formed readily due to the presence of a tertiary carbocation. However, this carbocation would be destabilized by the presence of the electron withdrawing nature of the carboxylic acid.

The low reactivity of methacrylic acid coupled with the lack of formation of 2-chloro-2-methyl propionic acid leads one to speculate that the Michael-type addition of chloride is the operating mechanism. The enolate generated from the addition of chloride to crotonic acid would be more stable than the enolate generated from methacrylic acid. The electron donating property of the alpha-methyl group in methacrylic acid would destabilize the enolate compared to crotonic acid. Cinnamic acid was unreactive when exposed to these conditions.

### EXAMPLE 1

### Production of 3-chloropropionic acid

Acrylic acid (72 g; 1 mol) was mixed with isopropanol (66 g, 1.1 mol; 1.1 eq). The mixture was cooled to <15° C. in an ice bath. Acetyl chloride (86.5 g, 1.1 mol; 1.1 eq) was added dropwise from an addition funnel over 4 hours while maintaining the temperature <20° C. The clear solution was stirred for one hour at <20° C. before warming to room temperature and stirring for 16 hours. The excess HCl was removed by a sub-surface nitrogen sparge. The pressure was reduced to 100 mm Hg and the pot temperature increased to 110° C. to remove isopropyl acetate. Hexane (200-m1) was added and the temperature reduced to 20-25° C. to induce crystallization. The slurry was further cooled to 5-10° C. before the product was isolated by filtration. The solid was dried for four hours in a 25° C. vacuum oven to provide 97.6 grams of dry product. The material was assayed by NMR at 95.8% (86.6% yield).

### EXAMPLE 2

### Production of 3-chloropropionamide

Acrylamide (35.5 g; 0.5 mol) was slurried in isopropanol (60 g; 1.0 mol; 2.0 eq). The mixture was cooled to <15° C. in an ice bath. Acetyl chloride (80 g; 1.0 mol; 2.0 eq) was added dropwise from an addition funnel over 2-4 hours while maintaining the temperature <20° C. The slurry for stirred for one hour at <20° C. before warming to room temperature and stirring for 16 hours. Hexane (100 mL) was added and the temperature reduced to 20-25° C. to induce crystallization. The solid was air dried on the funnel for one hour to provide 66.0 grams of dry product. The material was assayed by NMR at 76.4% (93.8% yield).

### EXAMPLE 3

### Production of 3-chloropropionitrile

Acrylonitrile (53 g; 1 mol) was mixed in isopropanol (66 g; 1.1 mol; 1.1 eq). The solution was cooled to <15° C. in an ice bath. Acetyl chloride (86.3 g; 1.1 mol; 1.1 eq) was added dropwise from an addition funnel over 2-4 hours while maintaining the temperature <20° C. The solution was stirred for one hour at <20° C. before warming to room temperature and stirring for 16 hours. The excess HCl was removed by a sub-surface nitrogen sparge. The co-product isopropyl acetate was removed under reduced pressure to provide 82.6 grams of crude 3-chloropropionitrile. An analysis of the crude product by gas chromatography measured a composition of 95.6% 3-chloropropionitrile and 2.8% 3-chloropropionamide, providing a yield of 88.2%.

### EXAMPLE 4

### Production of 3-chloro-2-methyl propionic acid

Methacrylic acid (21.5 g; 0.25 mol) was mixed in isopropanol (45 g; 0.75 mol; 3.0 eq). The solution was cooled to <15° C. in an ice bath. Acetyl chloride (58.83 g; 0.75 mol; 3.0 eq) was added dropwise from an addition funnel over 2-4 hours while maintaining the temperature <20° C. The solution was stirred for two hours at <15° C. before warming to room temperature and stirring for 16 hours. The excess HCl was removed by a sub-surface nitrogen sparge. The co-product isopropyl acetate was removed under reduced pressure to provide 26.8 grams of crude 3-chloro-2-methyl propionic acid. An analysis of the crude product by gas chromatography measured a composition of 77.5% 3-chloro-2-methyl propionic acid, 4.2% isopropyl 3-chloro-2-methyl propionic acid, and 18.2% methacrylic acid on a normalized basis. The product yield was calculated to be 66.3%.

### EXAMPLE 5

### Preparation of 3-chlorobutanoic acid

Crotonic acid (43 g; 0.5 mol) was slurried in isopropanol (45 g; 0.75 mol; 1.5 eq). The solution was cooled to <15° C. in an ice bath. Acetyl chloride (59.0 g; 0.75 mol; 1.5 eq) was added dropwise from an addition funnel over 2-4 hours while maintaining the temperature <20° C. The solution was stirred for two hours at <30° C. before warming to room temperature and stirring for 16 hours. The excess HCl was removed by a sub-surface nitrogen sparge. The co-product isopropyl acetate was removed under reduced pressure to provide 69.5 grams of crude 3-chlorobutanoic acid. An analysis of the crude product by gas chromatography measured a composition of 79.8% 3-chlorobutanoic acid, 13.9% isopropyl 3-chlorobutanoic acid, 4.7% isopropyl crotonate and 1.5% crotonic acid on a normalized basis. The product yield was calculated to be 76.2%.

### EXAMPLE 6

### Preparation of 3-chloro-2-methyl propionitrile

Methacrylonitrile (33.5 g; 0.5 mol) was dissolved in isopropanol (60 g; 1.0 mol; 1.0 eq). The solution was cooled to <15° C. in an ice bath. Acetyl chloride (78.5 g; 1.0 mol; 1.0 eq) was added dropwise from an addition funnel over 2-4 hours while maintaining the temperature <20° C. The solution was stirred for two hours at <20° C. before warming to room temperature and stirring for 16 hours. The excess HCl was removed by a sub-surface nitrogen sparge. The co-product isopropyl acetate was removed under reduced pressure to provide 58.3 grams of crude 3-chloro-2-methyl propionitrile. An analysis of the crude product by gas chromatography measured a composition of 99.7% 3-chloro-2-methyl propionitrile and 0.3% methyacrylonitrile on a normalized basis. The product yield was calculated to be 86.3%.

Lastly, the invention also includes all of the aforementioned products that are manufactured by the methods outlined above.

While the invention will be described with respect to various embodiments thereof, it will be understood by those skilled in the art that various changes in detail may be made therein without departing from the spirit, scope, and teaching of the invention. Accordingly, the invention herein disclosed is to be limited only as specified in the claims.

In the drawings and specification, there have been disclosed typical embodiments on the invention and, although specific terms have been employed, they have been used in a generic and descriptive sense only and not for purposes of limitation, the scope of the invention being set forth in the following claims.

That which is claimed is:

1. A method for the hydrochlorination of alkenes utilizing in-situ generation of HCl, the method comprising the steps of:
forming a reaction mixture, said mixture comprising
an alkene of the general formula 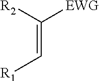
where R₁ is selected from the group consisting of H and CH₃, R₂ is selected from the group consisting of H and CH₃, and EWG is selected from the group consisting of COOH, CONH₂, and CN where R is an alkyl group having from 1 to 4 carbon atoms, provided that R₁ and R₂ are not both CH₃;
an alcohol selected from the group consisting of primary and secondary alcohols having 2 to 4 carbon atoms; and
an acid chloride of the general formula 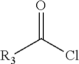
where R₃ is an alkyl group having 1 to 4 carbon atoms; and
maintaining said reaction mixture at a temperature sufficient to convert said alkene to a compound having the general formula

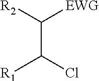

2. A method according to claim 1 wherein R₁ is H; R₂ is H; EWG is COOH, the alcohol is isopropanol, and the acid chloride is acetyl chloride.

3. A method according to claim 1 wherein R₁ is H; R₂ is H; EWG is CON H₂, the alcohol is isopropanol, and the acid chloride is acetyl chloride.

4. A method according to claim 1 wherein R₁ is H; R₂ is H; EWG is CN, the alcohol is isopropanol, and the acid chloride is acetyl chloride.

5. A method according to claim 1 wherein R₁ is CH₃, R₂ is H; EWG is COOH; the alcohol is isopropanol; and the acid chloride is acetyl chloride.

6. A method according to claim 1 wherein R₁ is H, R₂ is CH₃; EWG is COOH; the alcohol is isopropanol; and the acid chloride is acetyl chloride.

7. A method according to claim 1 wherein R₁ is H, R₂ is CH₃; EWG is CN; the alcohol is isopropanol; and the acid chloride is acetyl chloride.

8. A method according to claim 1 wherein the reaction mixture is non-aqueous.

9. A method for the hydrochlorination of alkenes utilizing in-situ generation of HCl, the method comprising the steps of:
forming a reaction mixture comprising
an alkene selected from the group consisting of acrylic acid, crotonic acid, methacrylic acid, acrylonitrile, methacrylonitrile, and acrylamide;
an alcohol selected from the group consisting of primary and secondary alcohols having 2 to 4 carbon atoms; and
an acid chloride; and
maintaining said reaction mixture at a temperature sufficient to chlorinate said alkene.

10. A method according to claim 9 wherein the acid chloride is acetyl chloride.

11. A method according to claim 10 wherein the alkene is acrylic acid, the alcohol is isopropanol, and further comprising the step of maintaining said reaction mixture at a temperature sufficient to chlorinate the acrylic acid to form 3-chloropropionic acid.

12. A method according to claim 10 wherein the alkene is crotonic acid, the alcohol is isopropanol, and further comprising the step of maintaining said reaction mixture at a temperature sufficient to chlorinate the acrylic acid to form 3-chlorobutonic acid.

14. A method according to claim 10 wherein the alkene is methacrylic acid, the alcohol is isopropanol, and further comprising the step of maintaining said reaction mixture at a temperature sufficient to chlorinate the methacrylic acid to form 3-chloro-2-methyl propionic acid.

15. A method according to claim 10 wherein the alkene is acrylonitrile, the alcohol is isopropanol, and further comprising the step of maintaining said reaction mixture at a temperature sufficient to chlorinate the acrylonitrile to form 3-chloropropionitrile.

16. A method according to claim 10 wherein the alkene is methacrylonitrile, the alcohol is isopropanol, and further comprising the step of maintaining said reaction mixture at a temperature sufficient to chlorinate the acrylonitrile to form 3-chloro-2-methyl propionitrile.

17. A method according to claim 10 wherein the alkene is acrylamide, the alcohol is isopropanol, and further comprising the step of maintaining said reaction mixture at a temperature sufficient to chlorinate the acrylamide to form 3-chloropropionamide.

18. A method according to claim 9 wherein the reaction mixture is non-aqueous.

19. A method for the hydrochlorination of alkenes utilizing in-situ generation of HCl, the method comprising the steps of:
forming a reaction mixture comprising
an alkene selected from the group consisting of acrylic acid, crotonic acid, methacrylic acid, acrylonitrile, methycrylonitrile, and acrylamide;
isopropanol; and
acetyl chloride; and
maintaining said reaction mixture at a temperature sufficient to chlorinate the alkene.

20. A chlorinated alkene made in accordance with the method of claim 9.
